(19) United States
(12) Patent Application Publication
(10) Pub. No.: US 2009/0169641 A1
(43) Pub. Date: Jul. 2, 2009

(54) COMPRESSIBLE PARTICLES

(75) Inventors: Goldi Kaul, Nyack, NY (US); Janel L. Lanphere, Flagstaff, AZ (US); Robert E. Richard, Wrentham, MA (US); Steve Keenan, Framingham, MA (US)

Correspondence Address: FISH & RICHARDSON PC, P.O. BOX 1022, MINNEAPOLIS, MN, 55440-1022, US

(73) Assignee: BOSTON SCIENTIFICE SCIMED, INC., Maple Grove, MN (US)

(21) Appl. No.: 12/337,400

(22) Filed: Dec. 17, 2008

Related U.S. Application Data
(60) Provisional application No. 61/016,615, filed on Dec. 26, 2007.

(51) Int. Cl. A61K 9/16; C08J 9/16; A61P 15/08

(52) U.S. Cl. 424/501; 521/50.5; 521/56

(57) ABSTRACT

Disclosed herein are methods that include providing a first particle having pores, the first particle having a first compression test value, and partially cross-linking the first particle to form a second particle having pores, the second particle having a second compression test value that is larger than the first compression test value by 25% or more, where a maximum dimension of the first particle is 5,000 microns or less, and a maximum dimension of the second particle is 5,000 microns or less.

### CROSS-REFERENCE TO RELATED APPLICATIONS

This application claims priority under 35 U.S.C. § 119, to U.S. Ser. No. 61/016,615, filed Dec. 26, 2007, the contents of which are hereby incorporated by reference.

### TECHNICAL FIELD

This disclosure relates to particles that can be delivered to sites within the body (e.g., embolic particles), as well as related compositions and methods.

### BACKGROUND

Particles can be delivered to sites within the body to perform various functions. For example, embolic particles can be placed within one or more body lumens.

### SUMMARY

In general, in a first aspect, the disclosure features a method that includes providing a first particle having pores, the first particle having a first compression test value, and partially cross-linking the first particle to form a second particle having pores, the second particle having a second compression test value that is larger than the first compression test value by 25% or more, where a maximum dimension of the first particle is 5,000 microns or less, and a maximum dimension of the second particle is 5,000 microns or less.

In another aspect, the disclosure features a method that includes: (a) providing a particle having pores and a maximum dimension of 5,000 microns or less, the particle having a first compression test value; (b) selecting a desired compression test value for the particle; and (c) changing the compression test value of the particle from the first compression test value to the second compression test value. The second compression test value can be at least 25% larger than the first compression test value.

In a further aspect, the disclosure features a method that includes: (a) providing a first plurality of particles having pores, and disposing a therapeutic agent in at least some of the pores of the first plurality of particles; and (b) partially cross-linking the first plurality of particles to form a second plurality of particles having pores. The first plurality of particles can have a first average compression test value, and the second plurality of particles can have a second average compression test value that is larger than the first average compression test value by 25% or more.

In another aspect, the disclosure features a method that includes irradiating a first porous particle having a first compression test value to form a second porous particle having a second compression test value, the second compression test value being larger than the first compression test value.

Embodiments can include one or more of the following features.

The second compression test value can be larger than the first compression test value by 50% or more (e.g., 100% or more, 200% or more).

Partially cross-linking the first particle can include irradiating the first particle. Irradiating the first particle can include irradiating the first particle with electrons. Alternatively, or in addition, irradiating the first particle can include irradiating the first particle with gamma radiation.

The first particle can include vinyl alcohol monomer units, and the method can include cross-linking at least some of the vinyl alcohol monomer units. The first particle can include vinyl formal monomer units, and the method can include cross-linking at least some of the vinyl formal monomer units. Alternatively, or in addition, cross-linking can also occur between chemical moieties in one or more polymer chains that form the particles, and/or in polyvinyl alcohol and/or polyvinyl acetal pendant groups attached to the chains.

A dosage of the electron radiation can be 10 kiloGray or more (e.g., 25 kiloGray or more, 50 kiloGray or more, 75 kiloGray or more).

The method can include, before partially cross-linking the first particle, disposing a therapeutic agent in at least some of the pores of the first particle.

The method can include forming a plurality of the second particles, and forming a composition comprising a carrier solution and the plurality of the second particles. The method can further include treating a subject with the composition.

Changing the compression test value of the particle can include partially cross-linking the particle to form a second particle having pores, where the second particle has the second compression test value.

The method can include, before changing the compression test value of the particle, disposing a therapeutic agent in at least some of the pores of the particle.

The second average compression test value can be larger than the first average compression test value by 50% or more (e.g., by 100% or more, by 200% or more).

The method can include forming a composition by combining the second plurality of particles with a carrier solution.

The second compression test value can be larger than the first compression test value by a factor of 2 or more (e.g., by a factor of 3 or more, by a factor of 5 or more, by a factor of 7 or more).

Embodiments can include one or more of the following advantages.

The particles can include one or more therapeutic agents, and the agents can be used for treatment of physiological conditions in a subject. Particle properties such as pore size and particle composition can be selected to control the rate of release of the one or more agents carried by the particles into the subject's body. Particles can also include targeting chemical moieties that assist in directing the particles to specific sites within the subject's body.

The particles can be used for embolization of body lumens. Particles can include adhesion moieties in particle pores. The adhesion moieties cause particles to adhere to one another, thereby increasing the efficiency of the embolization process.

Irradiating one or more polymer components of the particles with electrons or gamma radiation can cross-link the particles without substantially changing the size of the particles. In addition, the cross-linked particles can be less compressible (e.g., have a higher compression test value) than non-cross-linked particles. As a result, the particles form more predictable and efficient embolizations than un-cross-linked particles because the particles do not deform as severely in body lumens.

Changing the compressibility of the cross-linked particles via irradiation can permit control over the placement of the particles in body lumens. For example, altering the particle compressibility of the particles can control the (minimum) lumen size through which the particles can pass. By reducing the compressibility of the particles, inadvertent placement of the particles within certain small-bore lumens can be prevented, because the relatively less compressible particles are not able to deform sufficiently to pass through the small-bore lumens.

Irradiating one or more polymer components of the particles with electrons or gamma radiation can cross-link the particles without initiating chemical changes in one or more therapeutic agents carried by the particles. As a result, agents that are carried by the particles (e.g., in particle pores) remain in active form following the cross-linking procedure, and can be delivered to body sites by the particles, where the agents can perform various therapeutic functions.

Other features and advantages will be apparent from the description, drawings, and claims.

### DESCRIPTION OF DRAWINGS

FIGS. 1A and 1B are schematic diagrams of an un-cross-linked porous particle and a cross-linked porous particle, respectively.

FIGS. 2A, 2B, and 3 are schematic diagrams of an embodiment of a system and method for producing particles.

FIG. 4A is a schematic diagram of an embodiment of a method of injecting a composition including particles into a body lumen.

FIG. 4B is an enlarged view of region 4B in FIG. 4A.

FIG. 5 is a plot of compression force as a function of strain for various types of particles.

FIG. 6 is a plot of compression force as a function of strain for various types of particles.

FIG. 7 is a graph showing compression force at 80% strain for particles of different formulations measured by two different operators.

FIG. 8 is a graph showing compression force at 80% strain for different types of particles exposed to different doses of electron beam radiation.

FIG. 9 is a graph showing compression force at 80% strain for different types of particles in a nitrogen-free environment.

FIG. 10 is a graph showing compression force at 80% strain for different types of particles in an environment that includes nitrogen.

Like reference symbols in the various drawings indicate like elements.

### DETAILED DESCRIPTION

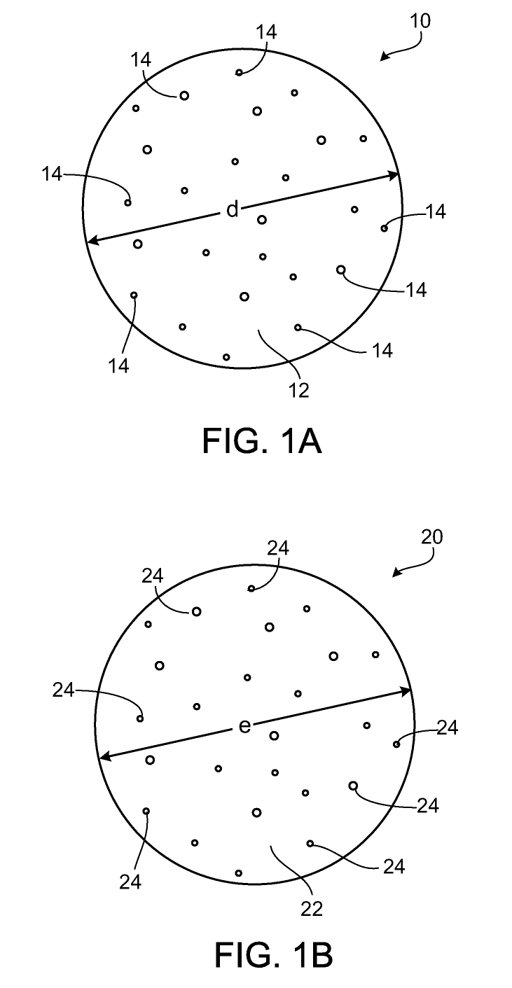

FIGS. 1A and 1B show, respectively, particles 10 and 20 that can be used, for example, in an embolization procedure. Particle 10 includes a matrix 12 and a plurality of pores 14, and has a maximum dimension d measured along a line that extends through a center of mass of particle 10.

Particle 20 includes a matrix 22 and a plurality of pores 24, and has a maximum dimension e measured along a line that extends through a center of mass of particle 20. Particle 20 is formed by cross-linking one or more constituents of particle 10 under a variety of environmental conditions. As a result, matrix 22 includes, at least in part, products of cross-linking one or more of the components of matrix 12.

In general, particle 10 can be formed from a variety of different materials. In some embodiments, for example, particle 10 includes one or more polymers formed from monomer units such as vinyl alcohol monomer units and/or vinyl formal monomer units (e.g., Formvar®, (available from Structure Probe Inc., West Chester, Pa.) and/or vinyl acetate monomer units. As referred to herein, a vinyl formal monomer unit has the following structure:

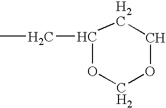

As referred to herein, a vinyl alcohol monomer unit has the following structure:

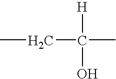

As referred to herein, a vinyl acetate monomer unit has the following structure:

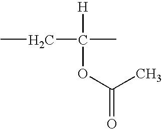

In general, the monomer units can be arranged in a variety of different ways. As an example, in some embodiments, polymers can include different monomer units that alternate with each other. For example, polymers can include repeating blocks, each block including a vinyl formal monomer unit, a vinyl alcohol monomer unit, and a vinyl acetate monomer unit. As another example, in certain embodiments, polymers can include blocks including multiple monomer units of the same type.

In some embodiments, polymers can have the formula that is schematically represented below, in which x, y and z each are integers that are greater than zero. In certain embodiments, x is zero. The individual monomer units that are shown can be directly attached to each other, and/or can include one or more other monomer units (e.g., vinyl formal monomer units, vinyl alcohol monomer units, vinyl acetate monomer units) between them:

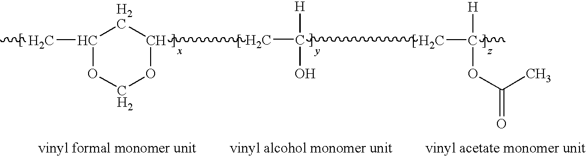

Optionally, formal linkages can occur between PVA monomers giving crosslinks.

In general, the weight percentages of the constituents of particle can be selected to control the mechanical properties of particle 20 after particle 10 is cross-linked, as discussed above. In some embodiments, polymers can include at least five percent by weight (e.g., at least 15 percent by weight, at least 25 percent by weight, at least 35 percent by weight) vinyl formal monomer units, and/or at most 80 percent by weight (e.g., at most 50 percent by weight, at most 25 percent by weight, at most 10 percent by weight) vinyl formal monomer units. The weight percent of a monomer unit in a polymer can be measured using solid-state NMR spectroscopy.

Generally, the polymer will contain relatively fewer vinyl alcohol monomer units than vinyl formal monomer units. In some embodiments, the polymer can include at most 10 percent by weight (e.g., at most 5 percent by weight, at most 2 percent by percent by weight) vinyl alcohol monomer units and/or at least 0.1 percent by weight (e.g., at least 0.5 percent by weight, at least 1 percent by weight) vinyl alcohol monomer units. As used herein, the weight percent of a monomer unit in a polymer is measured using solid-state NMR spectroscopy.

In some embodiments, the polymer can include at least one percent by weight (e.g., at least two percent by weight, at least five percent by weight, at least 10 percent by weight, at least 15 percent by weight) vinyl acetate monomer units, and/or at most 20 percent by weight (e.g., at most 15 percent by weight, at most 10 percent by weight, at most five percent by weight) vinyl acetate monomer units. As used herein, the weight percent of a monomer unit in a polymer is measured using solid-state NMR spectroscopy.

As a result of cross-linking particle 10, particle can include a variety of polymers in addition to, or in alternative to, the polymers disclosed above. Examples of polymers that can be constituents of particle 20 include polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids) and copolymers or mixtures thereof. Polymers suitable to form particle 20 are described, for example, in: Lanphere et al., U.S. Patent Application Publication No. US 2004/0096662, published on May 20, 2004, and entitled “Embolization”; Song et al., U.S. patent application Ser. No. 11/314,056, filed on Dec. 21, 2005, and entitled “Block Copolymer Particles”; and Song et al., U.S. patent application Ser. No. 11/314,557, filed on Dec. 21, 2005, and entitled “Block Copolymer Particles”. The entire contents of each of the foregoing applications are incorporated herein by reference.

Particle 10 can also be formed from one or more sacrificial materials that are removed from particle 10 prior to cross-linking to form pores 14 in matrix 12. Cross-linking leads to the formation of pores 24 from pores 14. Exemplary sacrificial materials that can be included in particle 10 include alginate, chitosan, chitin.

In general, the weight percentages of the one or more sacrificial materials in particle 10 can be selected to control the porosity of particle 10 when the one or more sacrificial materials are removed. In some embodiments, the weight percentage of a sacrificial material in particle 10 can be 5% or more (e.g., 10% or more, 15% or more, 20% or more). In certain embodiments, the weight percentage of a sacrificial material in particle 10 can be 30% or less (e.g., 20% or less, 10% or less, 5% or less).

The maximum dimension of particle 10 can, in general, be selected according to a particular intended function for particle 10 within the body. In some embodiments, d can be 5,000 microns or less (e.g., 4,500 microns or less, 4,000 microns or less, 3,500 microns or less, 3,000 microns or less, 2,500 microns or less, 2,000 microns or less, 1,500 microns or less, 1,200 microns or less, 1,150 microns or less, 1,100 microns or less, 1,050 microns or less, 1,000 microns or less, 900 microns or less, 700 microns or less, 500 microns or less, 400 microns or less, 300 microns or less, 100 microns or less, 50 microns or less, 10 microns or less, five microns or less) and/or one micron or more (e.g., five microns or more, 10 microns or more, 50 microns or more, 100 microns or more, 300 microns or more, 400 microns or more, 500 microns or more, 700 microns or more, 900 microns or more, 1,000 microns or more, 1,050 microns or more, 1,100 microns or more, 1,150 microns or more, 1,200 microns or more, 1,500 microns or more, 2,000 microns or more, 2,500 microns or more). In some embodiments, the maximum dimension d of particle 10 is less than 100 microns (e.g., less than 50 microns).

As discussed above, particle 10 is converted to particle 20 by at least partially cross-linking one or more polymer constituents of particle 10. In certain embodiments, one or more polymers in particle 10 can be cross-linked by irradiating particle 10 with gamma radiation. For example, in some embodiments, particle 10 can receive a dosage of 10 kGy or more (e.g., 20 kGy or more, 30 kGy or more, 50 kGy or more, 75 kGy or more) to cross-link one or more polymers in particle 10.

In some embodiments, one or more polymers in particle 10 can be cross-linked by irradiating particle 10 with electrons. For example, in certain embodiments, a dosage of electron beam radiation used to irradiate particle 10 can be 10 kGy or more (e.g., 15 kGy or more, 20 kGy or more, 25 kGy or more, 30 kGy or more, 40 kGy or more, 50 kGy or more, 75 kGy or more, 100 kGy or more, 125 kGy or more, 150 kGy or more, 175 kGy or more, 200 kGy or more).

In certain embodiments, one or more polymers in particle 10 can be cross-linked by temperature cycling. For example, in some embodiments, particle 10 can be maintained for a duration of time at a temperature of 15° C. or less (e.g., 10° C. or less, 0° C. or less, −15° C. or less, −25° C. or less, −35° C. or less, −50° C. or less, −60° C. or less). For example, particle 10 can be reduced to a temperature of from −80° C. to −50° C. (e.g., from −75° C. to −60° C., from −75° C. to −65° C.). In certain embodiments, particle can be maintained at a temperature of −70° C.

In certain embodiments, particle 10 can be exposed to one or more additional chemical agents during cross-linking of at least some of the polymer constituents of particle 10. Exposure of the polymer constituents of particle 10 to cross-linking conditions (e.g., including electron and/or gamma irradiation) causes some of the chemical bonds in the polymer constituents of particle 10 to break. The resulting bond fragments in particle 10 are available to form bonds with other fragments, leading to cross-linking of the polymer constituents as disclosed above. The bond fragments can also form new bonds with other chemical agents, thereby introducing new chemical moieties into the structure of particle 10. For example, in some embodiments, particle 10 can be exposed to additional chemical agents in the form of one or more reactive gases to introduce new chemical moieties into the structure of particle 10. Many different reactive gases can be used for this purpose including, for example, gases such as ammonia, arsine, boron trichloride, germanium tetrachloride, silicon tetrachloride, titanium tetrachloride, boron trifluoride, arsenic pentafluoride, phosphorus pentafluoride, tungsten hexafluoride, 1,3-butadiene, carbon monoxide, chlorine, diborane, dichlorosilane, germane, hydrogen bromide, hydrogen chloride, hydrogen cyanide, hydrogen fluoride, hydrogen selenide, hydrogen sulfide, nitric oxide, nitrogen dioxide, organoarsenic gases, organotin gases, organoindium gases, organogallium gases, phosgene, phosphine, silane, vinyl chloride, acetylene, hydrogen, isobutane, methane, nitrogen trifluoride, oxygen, propane, and sulfur hexafluoride. Derivatives of these gases can also be used, along with various other reactive gases.

In some embodiments, particle 10 can be exposed to additional chemical agents in the form of one or more reactive liquids to introduce new chemical moieties into the structure of particle 10 during cross-linking. Reactive liquids that can be used for this purpose include a variety of liquid substances with unsaturated chemical moieties, including, for example, hydroxyethyl methacrylates, hydroxyethyl acrylates, vinyl pyridines, vinyl sulfones, vinyl acetic acids, allyl amines, and allyl alcohols. In certain embodiments, when particle 10 is exposed to one or more reactive liquids, particle 10 can be maintained in an inert environment (e.g., by placing particle 10 in a sealed vessel that is first evacuated, and then backfilled with a relatively unreactive gas). Suitable gases for maintaining particle 10 in an inert environment include nitrogen and noble gases (e.g., argon), for example.

In certain embodiments, particle 10 can be exposed to additional chemical agents in the form of one or more polymers (e.g., monomers, dimers, trimers, oligomers) during cross-linking. The one or more polymers can bind and be cross-linked to one or more polymer components in particle 10. As a result of introducing these additional polymers, the physical properties of particle 20 which is produced from cross-linking particle 10 (e.g., compressibility, tensile strength, shear strength, elasticity) can be modified. Exemplary polymers that can be introduced into the structure of particle 10 during cross-linking include polyvinyl alcohols, polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids), and copolymers and/or mixtures thereof.

In particular, by controlling the cross-linking of one or more components of particle 10, the compression test value of the resulting particle 20 can be controlled. The compression test value of particle 20 refers to a force applied to particle 20 with a force measurement system to cause particle 20 to deform from its equilibrium shape. The deformation of particle 20 from its equilibrium shape is referred to as strain. Strain is expressed mathematically as a change in a length of particle 20 along a coordinate direction. In general, the maximum dimension e of particle 20 can change along a generalized coordinate direction denoted by u, so that the strain S in particle 20 can be written as S=de/du.

The compression test value of particle 20 is measured using a compression force measurement system. A suitable compression force measurement system for measuring particle 20 includes Texture Analyzer Model TA-XT Plus (Texture Technologies Corporation, Scarsdale, N.Y.), an Elmo CCD TV camera Model HC7501 (Elmo Manufacturing Corporation, Plainview, N.Y.), and a Nikon optical microscope Model SMZ-2B (Nikon USA Corporation, El Segundo, Calif.). Particle 20 is positioned between a pair of compression plates, and a compression force is applied to cause deformation of the particle. The strain in particle 20 is measured along an axis orthogonal to the plane surfaces of the compression plates, and is assessed using the texture analyzer, CCD camera, and optical microscope. When a desired strain value is reached (typically, from 0-80% strain), the force on the compression plates is measured. This measured force corresponds to the force necessary to induce the selected strain value in particle 20. In general, the compression properties of different types of particles are compared at particular strain values. Herein, the compression test value of a particle refers to the measured force required to induce 80% strain in the particle.

In some embodiments, particle 20 has a compression test value that is at least 10% larger than a compression test value of particle 10, from which particle 20 is formed. For example, in certain embodiments, the compression test value of particle 20 is at least 15% larger (e.g., at least 20% larger, at least 25% larger, at least 50% larger, at least 100% larger, at least 200% larger, at least 300% larger, at least 400% larger, at least 500% larger, at least 600% larger, at least 700% larger, at least 800% larger) than the compression test value of particle 10.

In certain embodiments, particle 20 has a compression test value that is larger than a compression test value of particle 10 by a factor of 1.25 or more (e.g., by a factor of 1.5 or more, by a factor of 1.75 or more, by a factor of 2 or more, by a factor of 2.5 or more, by a factor of 3 or more, by a factor of 4 or more, by a factor of 5 or more, by a factor of 6 or more, by a factor of 7 or more).

In certain embodiments, particle 10 can be formed according to methods disclosed in Lanphere et al., U.S. Patent Application Publication No. US 2003/0185895 entitled “Drug Delivery Particle”, filed on Aug. 30, 2002, the contents of which are incorporated herein by reference. A solution containing a polyol and a gelling precursor such as sodium alginate can be delivered to a viscosity controller, which heats the solution to reduce its viscosity prior to delivery to a drop generator. The drop generator forms and directs drops into a gelling solution containing a gelling agent which interacts with the gelling precursor. For example, in the case where an alginate gelling precursor is employed, an agent containing a divalent metal cation such as calcium chloride can be used as a gelling agent, which stabilizes the drops by gel formation based on ionic crosslinking. A pore structure in the center of the particle has been observed to form in the gelling stage. The concentration of the gelling agent can control void formation in the particle, thereby controlling the porosity gradient in the particle. Adding non-gelling ions, for example, sodium ions, to the gelling solution can limit the porosity gradient, resulting in a more uniform intermediate porosity throughout the particle. The gel-stabilized drops can then be transferred to a reactor vessel, where the polymers in the gel-stabilized drops are reacted, thereby forming precursor particles. For example, the reactor vessel can include an agent that chemically reacts with the polyol to cause interchain or intrachain crosslinking. For example, the vessel can include an aldehyde and an acid, leading to acetalization of the polyol. The precursor particles are then transferred to a gel dissolution chamber, where the gel is dissolved. For example, ionically crosslinked alginate can be removed by ion exchange with a solution of sodium hexa-metaphosphate. Alginate can also be removed by radiation degradation. The particles can then be filtered to remove any residual debris and to sort the particles into desired size ranges. The filtered particles can then be sterilized (e.g., by electron beam irradiation) and packaged, typically in saline. Although chemical crosslinking is described in Lanphere et al., other crosslinking techniques such as crosslinking by irradiation and/or by repeated freezing and thawing can also be employed.

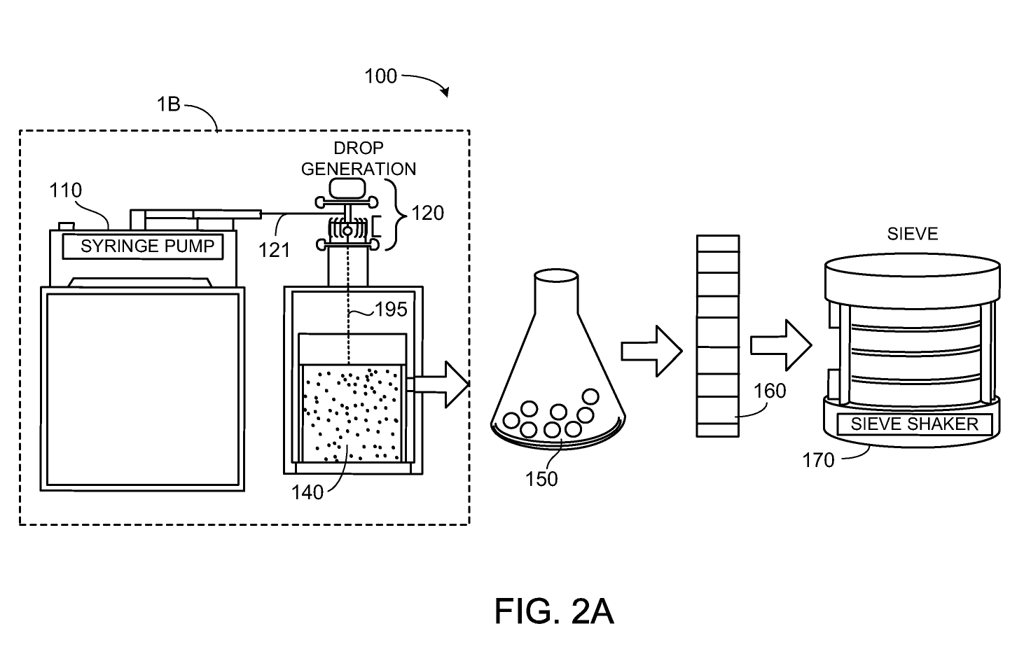

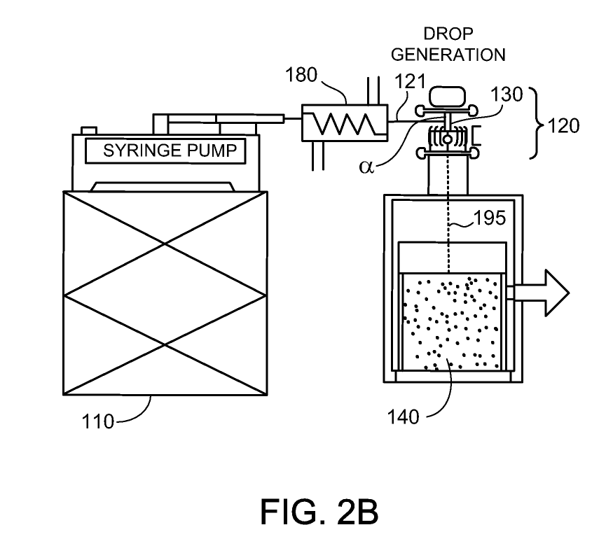

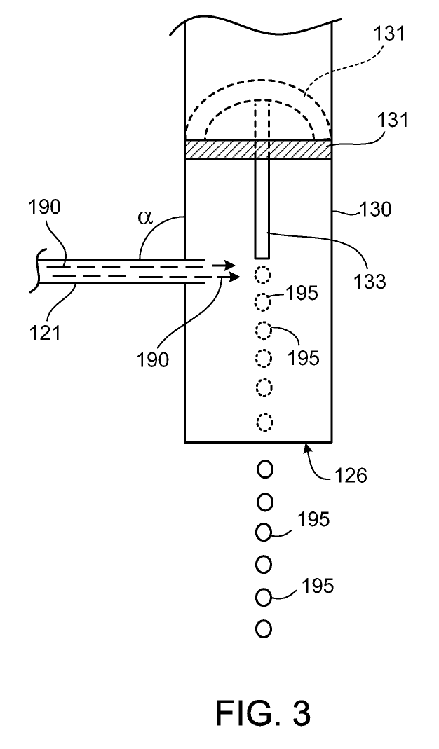

FIGS. 2A, 2B, and 3 show a system 100 for producing a plurality of particles 10. System 100 includes a flow controller 110, a drop generator 120 including a nozzle 130, a gelling vessel 140, a cooling vessel 150, an optional gel dissolution chamber 160, and a filter 170. An example of a commercially available drop generator is the model NISCO Encapsulation unit VAR D (NISCO Engineering, Zurich, Switzerland).

Flow controller 110 includes a high pressure pumping apparatus, such as a syringe pump (e.g., model PHD4400, Harvard Apparatus, Holliston, Mass.). Flow controller 110 delivers a stream 190 of a solution including a polymer (e.g., poly(vinyl alcohol) and/or poly(vinyl formal)) and a gelling precursor to a viscosity controller 180, which heats the solution to reduce its viscosity prior to delivery to drop generator 120. Viscosity controller 180 is connected to nozzle 130 of drop generator 120 via tubing 121. After stream 190 has traveled from flow controller 180 through tubing 121, stream 190 flows around a corner having an angle α, and enters nozzle 130. As shown, angle α is about 90 degrees. However, in some embodiments, angle α can be less than 90 degrees (e.g., less than about 70 degrees, less than about 50 degrees, less than about 30 degrees).

As stream 190 enters nozzle 130, a membrane 131 in nozzle 130 is subjected to a periodic disturbance (a vibration). The vibration causes membrane 131 to pulse upward (to the position shown in phantom in FIG. 3) and then return back to its original position. Membrane 131 is connected to a rod 133 that transmits the vibration of membrane 131, thereby periodically disrupting the flow of stream 190 as stream 190 enters nozzle 130. This periodic disruption of stream 190 causes stream 190 to form drops 195. Drops 195 fall into gelling vessel 140, where drops 195 are stabilized by gel formation. During gel formation, the gelling precursor in drops 195 is converted from a solution to a gel form by a gelling agent contained in gelling vessel 140. The gel-stabilized drops are then transferred from gelling vessel 140 to cooling vessel 150, where the polymer in the gel-stabilized drops is cooled and maintained at a reduced temperature to allow at least partial crystallization of the polymer to form particles 10. The particles are subsequently thawed. The cooling/thawing cycle can be repeated as desired to obtain, for example, a desired degree of crystallinity of the polymer.

In some embodiments, when in cooling vessel 150, particles 10 are reduced to a temperature less than 15° C. (e.g., less than 10° C., less than 0° C., less than −15° C., less than −25° C., less than −35° C., less than −50° C., less than −60° C.). For example, when in cooling vessel 150, the particles are reduced to a temperature of from −80° C. to −50° C. (e.g., from −75° C. to −60° C., from −75° C. to −65° C.). In certain embodiments, when in cooling vessel 150, the particles are at a temperature of −70° C.

In certain embodiments, particles 10 are held in cooling vessel 150 at reduced temperature for at least 10 minutes (e.g., at least 30 minutes, at least one hour, at least two hours, at least five hours, at least 10 hours, at least 20 hours, at least one day) and/or at most one week (e.g., at most three days, at most two days, at most one day). For example, the particles can be held in cooling vessel 150 at reduced temperature for from one hour to two days (e.g., from five hours to two days, from 10 hours to two days). In some embodiments, the particles are held in cooling vessel 150 at reduced temperature for one day.

In some embodiments, when thawing particles 10, the temperature of the particles is increased to at least 10° C. (e.g., at least 20° C., at least 25° C.). For example, when thawing the particles, the temperature of the particles can be increased to a temperature from 10° C. to 30° C. (e.g., from 15° C. to 30° C., from 20° C. to 30° C.). In certain embodiments, when thawing the particles, the particles are at a temperature of 25° C.

In certain embodiments, particles 10 are held at the relatively high (e.g., from 10° C. to 30° C.) temperature for at least 10 minutes (e.g., at least 30 minutes, at least one hour, at least two hours, at least three hours, at least four hours, at least five hours, at least six hours) and/or at most one week (e.g., at most three days, at most one day, at most 15 hours, at most 10 hours). For example, the particles are held at the relatively high (e.g., from 10° C. to 30° C.) temperature for from one hour to one day (e.g., from two hours to 10 hours, from four hours to 10 hours). In some embodiments, the particles are held at the relatively high (e.g., from 10° C. to 30° C.) temperature for six hours.

In some embodiments, the cooling/thawing cycle is repeated at least two times (e.g., at least three times, at least four times, at least five times, at least six times) and/or at most 100 times (e.g., at most 50 times, at most 25 times, at most 10 times). For example, the cooling/thawing cycle can be repeated from two times to 25 times (e.g., from four times to 10 times, from five times to 10 times). In certain embodiments, the cooling/thawing cycle is repeated six times.

In some embodiments, the cooling/thawing process is as follows: particles 10 are held in cooling vessel 150 at a temperature of less than −50° C. (e.g., from −80° C. to −60° C.) for at least 10 hours (e.g., from 10 hours to two day); the particles are then held at a temperature of at least 15° C. (e.g., from 20° C. to 30° C.) for at least two hours (e.g., from four hours to 10 hours); and the cooling/thawing cycle is repeated at least two times (e.g., from three times to six times).

Optionally, in addition to being disposed in cooling vessel 150 at the temperatures noted above, particles 10 can be disposed in a coolant (e.g., liquid nitrogen, liquid carbon dioxide) for a period of time (e.g., one minute to one hour, two minutes to 30 minutes, three minutes to 10 minutes, five minutes).

After the cooling/thawing cycle(s), particles 10 can optionally be transferred to gel dissolution chamber 160. In gel dissolution chamber 160, the gelling precursor (which was converted to a gel) in the particles is dissolved. After the matrix formation process has been completed, the particles can be filtered in filter 170 to remove debris, and sterilized. Particles 10 can then be cross-linked using any of the methods disclosed above to generate a plurality of particles 20.

Drop generators are described, for example, in Lanphere et al., U.S. Patent Application Publication No. US 2004/0096662, published on May 20, 2004, and entitled “Embolization”, and in DiCarlo et al., U.S. patent application Ser. No. 11/111,511, filed on Apr. 21, 2005, and entitled “Particles”, both of which are incorporated herein by reference.

Particles 20 can be used to perform a variety of functions within body lumens. In some embodiments, for example, particles 20 can be used to occlude a lumen in an embolization procedure. In certain embodiments, in addition to occluding a lumen, particles 20 can also include one or more therapeutic agents in pores 24. As a result, particles 20 can provide local chemotherapeutic action at body sites by delivering and releasing the therapeutic agent(s) to the sites. In some embodiments, for example, one or more therapeutic agents can be slowly released from pores 24 over time via diffusion following localization of the particles 20 at a body site.

A large variety of different therapeutic agents can be included in pores 24 for delivery by particles 20 to sites within the body. In some embodiments, for example, one or more therapeutic agents can be bonded within pores 24 to one or more polymer components of particles 20.

In general, therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶Ho), phosphorus (³²P), (¹⁷⁷Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). In some embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), and/or rhodium (¹⁰⁵Rh) can be used as therapeutic agents. In certain embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2 anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (IDEC pharmaceuticals, San Diego, Calif.) and Bexxar™ (Corixa corporation, Seattle, Wash.). Further examples of radioisotope/antibody pairs can be found in J. Nucl. Med. 2003, April: 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties, such as gelatin, chitosan, and collagen); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them.

Vectors of interest for delivery of genetic therapeutic agents include: plasmids; viral vectors such as adenovirus (AV), adenoassociated virus (AAV) and lentivirus; and non-viral vectors such as lipids, liposomes, and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

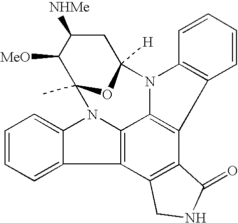

as well as diindoloalkaloids having one of the following general structures:

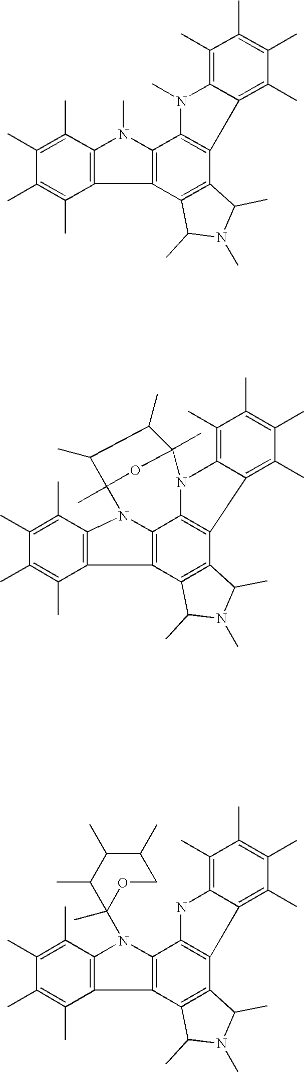

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Iib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include taxane derivatives (e.g., paclitaxel) and steroidal materials (e.g., dexamethasone).

Therapeutic agents are described, for example, in DiMatteo et al., U.S. Patent Application Publication No. US 2004/0076582 A1, published on Apr. 22, 2004, and entitled “Agent Delivery Particle”; Schwarz et al., U.S. Pat. No. 6,368,658; Buiser et al., U.S. patent application Ser. No. 11/311,617, filed on Dec. 19, 2005, and entitled “Coils”; and Song, U.S. patent application Ser. No. 11/355,301, filed on Feb. 15, 2006, and entitled “Block Copolymer Particles”, all of which are incorporated herein by reference.

Particles 20 can also include other materials. For example, pores 24 of particles 20 can include one or more radiopaque materials to increase the visibility of particles 20 in x-ray fluorescence imaging measurements. A radiopaque material can be, for example, a metal (e.g., tungsten, tantalum, platinum, palladium, lead, gold, titanium, silver), a metal alloy (e.g., stainless steel, an alloy of tungsten, an alloy of tantalum, an alloy of platinum, an alloy of palladium, an alloy of lead, an alloy of gold, an alloy of titanium, an alloy of silver), a metal oxide (e.g., titanium dioxide, zirconium oxide, aluminum oxide), bismuth subcarbonate, or barium sulfate. In some embodiments, a radiopaque material is a radiopaque contrast agent. Examples of radiopaque contrast agents include Omnipaque™, Renocal®, iodiamide meglumine, diatrizoate meglumine, ipodate calcium, ipodate sodium, iodamide sodium, iothalamate sodium, iopamidol, and metrizamide. Radiopaque contrast agents are commercially available from, for example, Bracco Diagnostic.

In some embodiments, pores 24 can include one or more MRI-visible materials for enhancing visibility of particles 20 in MRI measurements. An MRI-visible material can be, for example, a non-ferrous metal-alloy containing paramagnetic elements (e.g., dysprosium or gadolinium) such as terbium-dysprosium, dysprosium, and gadolinium; a non-ferrous metallic band coated with an oxide or a carbide layer of dysprosium or gadolinium (e.g., Dy₂O₃ or Gd₂O₃); a non-ferrous metal (e.g., copper, silver, platinum, or gold) coated with a layer of superparamagnetic material, such as nanocrystalline Fe₃O₄, CoFe₂O₄, MnFe₂O₄, or MgFe₂O₄; or nanocrystalline particles of the transition metal oxides (e.g., oxides of Fe, Co, Ni). In certain embodiments, an MRI-visible material can be an MRI contrast agent. Examples of MRI contrast agents include superparamagnetic iron oxides (e.g., ferumoxides, ferucarbotran, ferumoxsil, ferumoxtran (e.g., ferumoxtran-10), PEG-feron, ferucarbotran); gadopentetate dimeglumine; gadoterate meglumine; gadodiamide; gadoteridol; gadoversetamide; gadobutrol; gadobenate dimeglumine; mangafodipir trisodium; gadoxetic acid; gadobenate dimeglumine; macromolecular Gd-DOTA derivate; gadobenate dimeglumine; gadopentetate dimeglumine; ferric ammonium citrate; manganese chloride; manganese-loaded zeolite; ferristene; perfluoro-octylbromide; and barium sulfate. MRI contrast agents are described, for example, in U.S. patent application Ser. No. 10/390,202, now U.S. Publication No. US 2004/0186377, filed on Mar. 17, 2003 and entitled “Medical Devices”, the entire contents of which are incorporated herein by reference.

Radiopaque materials, MRI-visible materials, ferromagnetic materials, and contrast agents, any or all of which can be included in the pores of particles 20, are described, for example, in Rioux et al., U.S. Patent Application Publication No. US 2004/0101564 A1, published on May 27, 2004, and entitled “Embolization”, the entire contents of which are incorporated herein by reference.

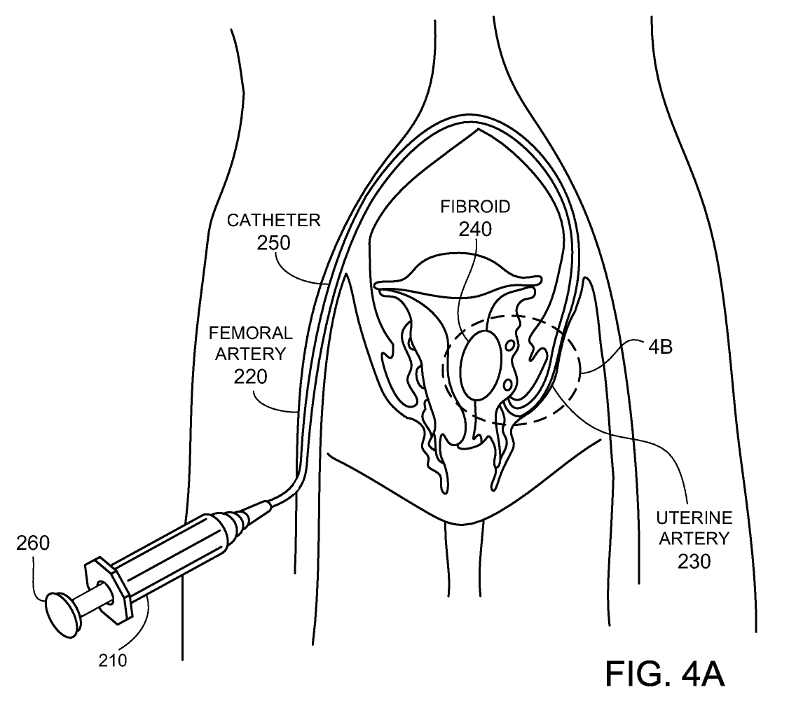

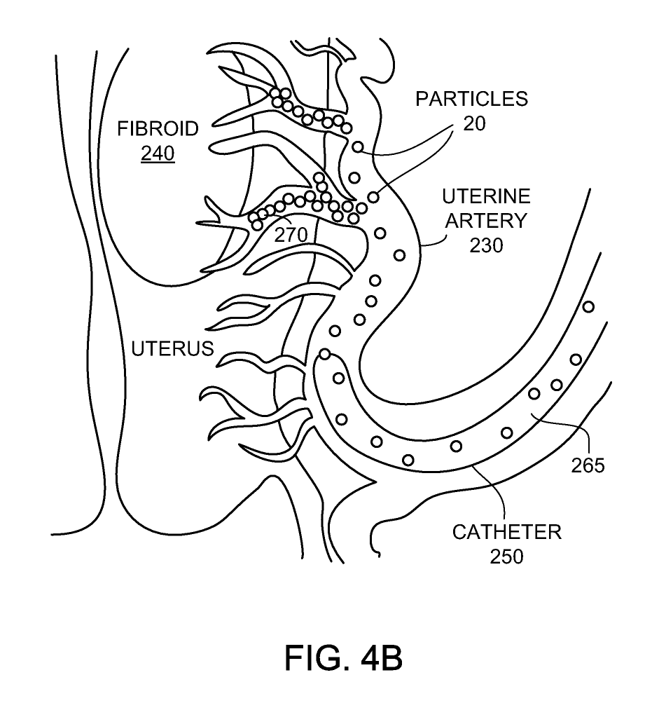

For therapeutic use, particles 20 can be introduced into the body intravenously, or more generally by injection into one or more body lumens. Particles 20 can be combined with a carrier fluid (e.g., a pharmaceutically acceptable carrier, such as a saline solution, a contrast agent, or both) to form a composition, which can then be delivered to a body site and used to embolize the site and/or to deliver one or more therapeutic agents to the site. FIGS. 4A and 4B illustrate the use of a composition including particles 20 to embolize a lumen of a subject. As shown, a composition including particles 20 and a carrier fluid is injected into a vessel through an instrument such as a catheter 250. Catheter 250 is connected to a syringe barrel 210 with a plunger 260. Catheter 250 is inserted, for example, into a femoral artery 220 of a subject. Catheter 250 delivers the composition to, for example, occlude a uterine artery 230 leading to a fibroid 240 located in the uterus of a female subject. The composition is initially loaded into syringe 210. Plunger 260 of syringe 210 is then compressed to deliver the composition through catheter 250 into a lumen 265 of uterine artery 230.

FIG. 4B, which is an enlarged view of region 4B of FIG. 4A, shows uterine artery 230, which is subdivided into smaller uterine vessels 270 (e.g., having a diameter of two millimeters or less) that feed fibroid 240. The particles 20 in the composition partially or totally fill the lumen of uterine artery 230, either partially or completely occluding the lumen of the uterine artery 230 that feeds uterine fibroid 240. Particles 20 can deliver one or more therapeutic agents into a region around fibroid 240.

Compositions including particles such as particles 20 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of particles 20 (e.g., particles formed of polymers including different weight percents of synthetic and nonsynthetic polymers, particles including different types of targeting moieties, adhesion moieties, therapeutic agents), or can include particles 20 that are all of the same type. In some embodiments, a composition can be prepared with a calibrated concentration of particles 20 for ease of delivery by a physician. A physician can select a composition of a particular concentration based on, for example, the type of procedure to be performed. In certain embodiments, a physician can use a composition with a relatively high concentration of particles 20 during one part of an embolization procedure, and a composition with a relatively low concentration of particles 20 during another part of the embolization procedure.

Suspensions of particles 20 in saline solution can be prepared to remain stable (e.g., to remain suspended in solution and not settle and/or float) over a desired period of time. A suspension of particles 20 can be stable, for example, for from one minute to 20 minutes (e.g. from one minute to 10 minutes, from two minutes to seven minutes, from three minutes to six minutes).

In some embodiments, particles 20 can be suspended in a physiological solution by matching the density of the solution to the density of the particles. In certain embodiments, particles 20 and/or the physiological solution can have a density of from one gram per cubic centimeter to 1.5 grams per cubic centimeter (e.g., from 1.2 grams per cubic centimeter to 1.4 grams per cubic centimeter, from 1.2 grams per cubic centimeter to 1.3 grams per cubic centimeter).

In certain embodiments, the carrier fluid of a composition can include a surfactant. The surfactant can help particles 20 to mix evenly in the carrier fluid and/or can decrease the likelihood of the occlusion of a delivery device (e.g., a catheter) by particles 20. In certain embodiments, the surfactant can enhance delivery of the composition (e.g., by enhancing the wetting properties of particles 20 and facilitating the passage of the particles through a delivery device). In some embodiments, the surfactant can decrease the occurrence of air entrapment by particles 20 in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from 0.05 percent by weight to one percent by weight (e.g., 0.1 percent by weight, 0.5 percent by weight) of a surfactant. A surfactant can be added to the carrier fluid prior to mixing with particles 20 and/or can be added to the particles prior to mixing with the carrier fluid.

In some embodiments, among a plurality of particles 20 delivered to a subject (e.g., in a composition), the majority (e.g., 50 percent or more, 60 percent or more, 70 percent or more, 80 percent or more, 90 percent or more) of the particles can have a maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles 10 delivered to a subject, the majority of the particles can have a maximum dimension of less than 100 microns (e.g., less than 50 microns).

In certain embodiments, particles 20 delivered to a subject (e.g., in a composition) can have an arithmetic mean maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, particles 10 delivered to a subject can have an arithmetic mean maximum dimension of less than 100 microns (e.g., less than 50 microns).

Exemplary ranges for the arithmetic mean maximum dimension of particles 20 delivered to a subject include from 100 microns to 500 microns; from 100 microns to 300 microns; from 300 microns to 500 microns; from 500 microns to 700 microns; from 700 microns to 900 microns; from 900 microns to 1,200 microns; and from 1,000 microns to 1,200 microns. In general, particles 20 delivered to a subject (e.g., in a composition) can have an arithmetic mean maximum dimension in approximately the middle of the range of the diameters of the individual particles, and a variance of 20 percent or less (e.g. 15 percent or less, 10 percent or less).

In some embodiments, the arithmetic mean maximum dimension of particles 20 delivered to a subject (e.g., in a composition) can vary depending upon the particular condition to be treated. As an example, in certain embodiments in which particles 20 are used to embolize a liver tumor, the particles delivered to the subject can have an arithmetic mean maximum dimension of 500 microns or less (e.g., from 100 microns to 300 microns; from 300 microns to 500 microns). As another example, in some embodiments in which particles 20 are used to embolize a uterine fibroid, the particles delivered to the subject can have an arithmetic mean maximum dimension of 1,200 microns or less (e.g., from 500 microns to 700 microns; from 700 microns to 900 microns; from 900 microns to 1,200 microns). As an additional example, in certain embodiments in which particles 20 are used to treat a neural condition (e.g., a brain tumor) and/or head trauma (e.g., bleeding in the head), the particles delivered to the subject can have an arithmetic mean maximum dimension of less than 100 microns (e.g., less than 50 microns). As a further example, in some embodiments in which particles 20 are used to treat a lung condition, the particles delivered to the subject can have an arithmetic mean maximum dimension of less than 100 microns (e.g., less than 50 microns). As yet another example, in certain embodiments in which particles 20 are used to treat thyroid cancer, the particles can have an arithmetic mean maximum dimension of 1,200 microns or less (e.g., from 1,000 microns to 800 microns). As a still further example, in some embodiments in which particles 20 are used only for therapeutic agent delivery, the particles can have an arithmetic mean maximum dimension of less than 100 microns (e.g., less than 50 microns, less than 10 microns, less than five microns).

In some embodiments, particle 20 can be substantially spherical. In certain embodiments, particle 20 can have a sphericity of 0.8 or more (e.g., 0.85 or more, 0.9 or more, 0.95 or more, 0.97 or more). Particle 20 can be, for example, manually compressed, essentially flattened, while wet to 50 percent or less of its original diameter and then, upon exposure to fluid, regain a sphericity of 0.8 or more (e.g., 0.85 or more, 0.9 or more, 0.95 or more, 0.97 or more). The sphericity of a particle can be determined using a Backman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Dₐ/Dₚ (where Dₐ=\sqrt{(4A/π)}; Dₚ=P/π; A=pixel area; P=pixel perimeter), is a value from zero to one, with one representing a perfect circle.

The arithmetic mean maximum dimension of a group of particles 20 can also be determined using a Beckman Coulter RapidVUE Image Analyzer. The arithmetic mean maximum dimension of a group of particles 20 (e.g., in a composition) can be determined by dividing the sum of the maximum dimensions of all of the particles 20 in the group by the number of particles in the group.

### EXAMPLES

A first group of particles was prepared according to the methods disclosed above from a mixture of components that included 10.5 weight percent poly(vinyl alcohol) and 1.5 weight percent sodium alginate, and the sodium alginate was subsequently removed to form pores in the particles. These particles will be referred to subsequently as Type 1 particles. A second group of particles was prepared using a similar procedure from a mixture of components that included 10.5 weight percent poly(vinyl alcohol) and 0.75 weight percent sodium alginate, and the sodium alginate was removed to form pores in the particles. These particles will be referred to subsequently as Type 2 particles. Relative to the Type 1 particles, Type 2 particles were formed from a mixture that included less sodium alginate (a sacrificial material) and therefore, the Type 2 particles had fewer pores, on average, than the Type 1 particles. The mean diameter of each of the groups of Type 1 and Type 2 particles was between 500 μm and 700 μm.

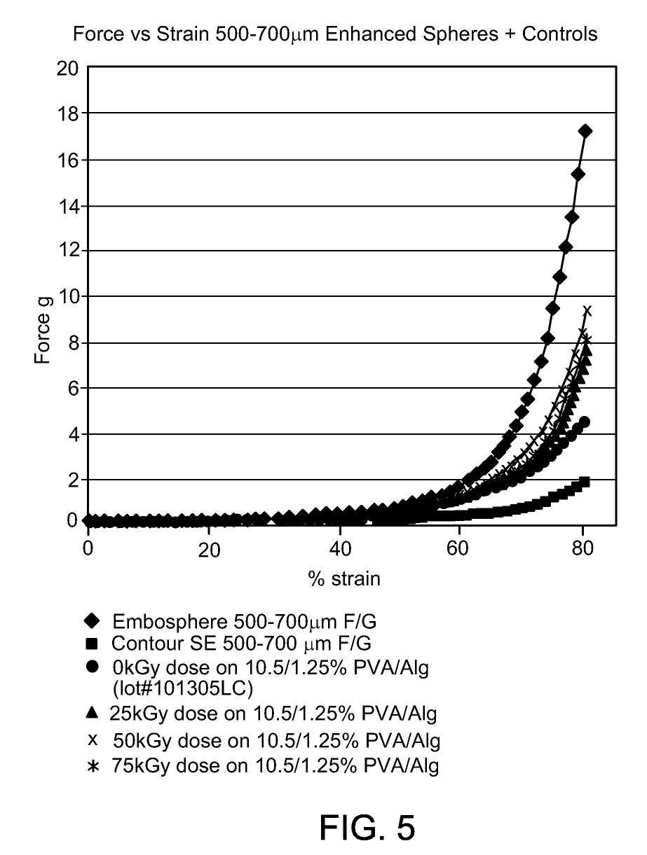

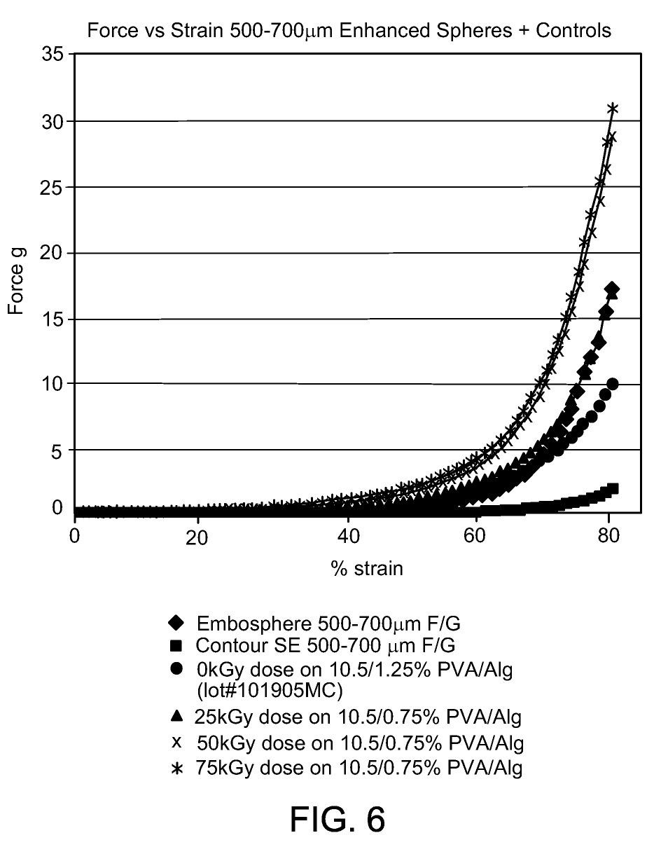

Samples of each of the Type 1 and Type 2 particles were exposed to different doses of electron beam radiation: 0 kGy, 25 kGy, 50 kGy, or 75 kGy. The compression force at a variety of strain values was determined for each type of particle using the compression force measurement system described above. Compression force measurements as a function of strain for Type 1 and Type 2 particles are shown in FIGS. 5 and 6, respectively. Referring to FIG. 5, differences in compression force among particles were more pronounced at higher strain values (e.g., from about 60% strain to about 80% strain). In particular, the compression force required to achieve 80% strain was higher by about 100% (e.g., a factor of about 2) after electron beam doses of 25 kGy, 50 kGy, and 75 kGy than with no electron beam radiation dose (0 kGy). However, the differences between the measured compression force to achieve 80% strain among particles exposed to doses of 25 kGy, 50 kGy, and 75 kGy were not as large. Compression force measurements for two other types of particles (Embosphere and Contour SE) are shown for comparative purposes.

Referring to FIG. 6, it was observed again that differences in compression force among the various Type 2 particles were more pronounced at higher strain values. The compression force required to achieve 80% strain was higher after an electron beam dose of 25 kGy by about 70% (e.g., a factor of about 1.7), and even higher after a dose of 50 kGy or 75 kGy by about 200% (e.g., a factor of about 3), than with no electron beam radiation dose (0 kGy). After a dose of 50 kGy or 75 kGy, the compression force of the Type 2 particles was significantly larger than the measured compression force of the particles after a dose of 25 kGy. As in FIG. 5, compression force measurements for Embosphere and Contour SE particles are shown for comparative purposes. Type 2 particles that received an electron beam radiation dose of 50 kGy or 75 kGy had significantly larger measured compression forces at 80% strain than either the Embosphere or Contour SE particles.

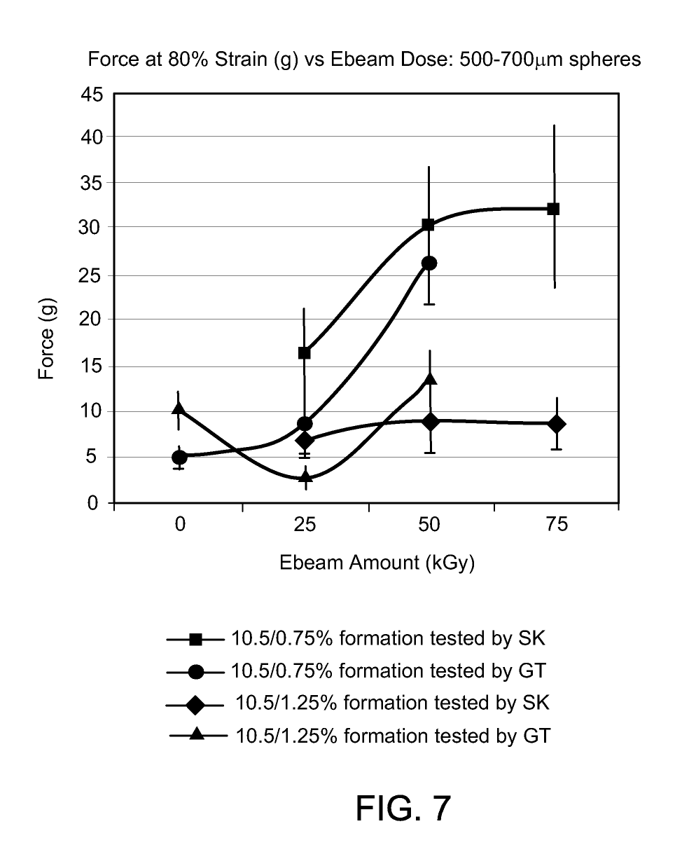

Compression force measurement results for the Type 1 and Type 2 particles are summarized in FIG. 7. The plot in FIG. 7 shows measurements for Type 1 particles (“10.5/1.25%”) and Type 2 particles (“10.5/0.75%”) independently performed by two different operators (“SK” and “GT”) at two different times. The force measurements from one operator do not numerically match the measurements from the other operator. However, the numerical compression force values measured by each operator follow a similar trend. Each operator's results indicate that the increase in compression force at 80% strain after a particular radiation dose for the Type 2 particles is larger than the increase in compression force after a particular radiation dose for the Type 1 particles. That is, the change in the measured compression force for a particular radiation dose is larger for the less-porous Type 2 particles than for the more-porous Type 1 particles. For the measurements performed by operator GT shown in FIG. 7, the compression test value of the Type 1 particles was increased by about 30% (e.g., a factor of about 1.3) via exposure of the particles to the electron beam. The compression test value of the Type 2 particles was increased by about 400% (e.g., a factor of about 5) via exposure of the particles to the electron beam.

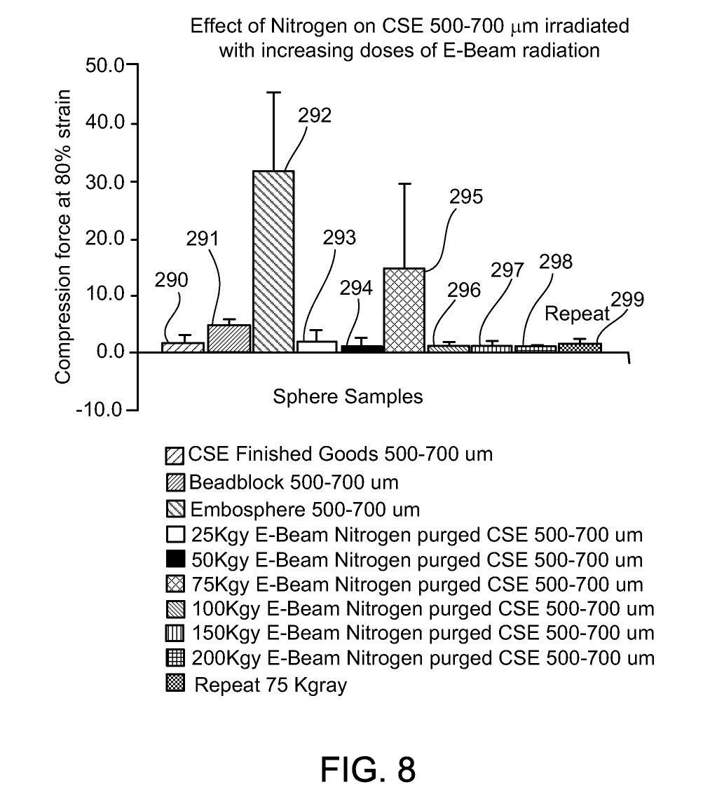

Compression force measurements were also performed on groups of Contour SE particles that were exposed to varying doses of electron beam radiation. Measured values of compression force at 80% strain are shown in FIG. 8 for a variety of different particles formed in a nitrogen-purged environment. The compression force for standard Contour SE particles is indicated at block 290. For reference purposes, blocks 291 and 292 indicate compression force measurements for Beadblock and Embosphere particles. Blocks 293-298 indicate measured compression force values for Contour SE particles exposed to the following electron beam radiation doses, respectively: 25 kGy, 50 kGy, 75 kGy, 100 kGy, 150 kGy, and 200 kGy. Block 299 indicates the measured compression force value after the particles that were initially exposed to a 75 kGy dose were exposed to a second 75 kGy dose. No readily identifiable trend was observed among the measured compression force values. Apparently, for certain types of particles, cross-linking constituents thereof may only produce higher compression forces if the particles are not too porous. Contour SE particles, in general, are more porous than either the Type 1 or Type 2 particles. Further, for the Contour SE particles, the compression force at 80% strain did not appear to be strongly influenced by the timing of radiation exposure. The compression force value for particles exposed to a single dose of electron beam radiation at 150 kGy (block 297) was similar to the compression force value for particles exposed to two doses of electron beam radiation of 75 kGy each (block 299). Based on the results shown in FIG. 8, the largest increase in compression test value was observed between block 290 (standard Contour SE particles) and block 295 (75 kGy radiation dose), and corresponded to an increase of about 600% to 700% (e.g., a factor of 7 to 8).

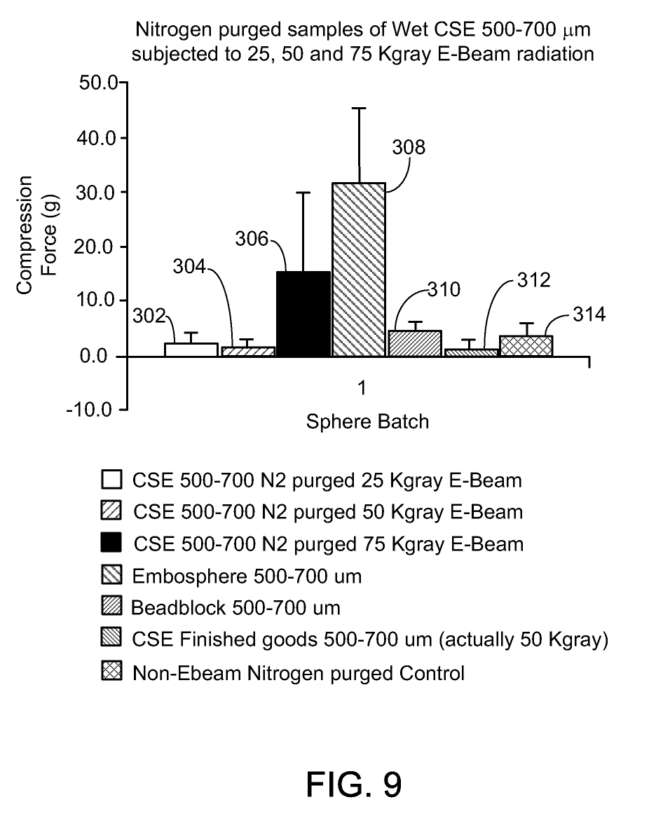

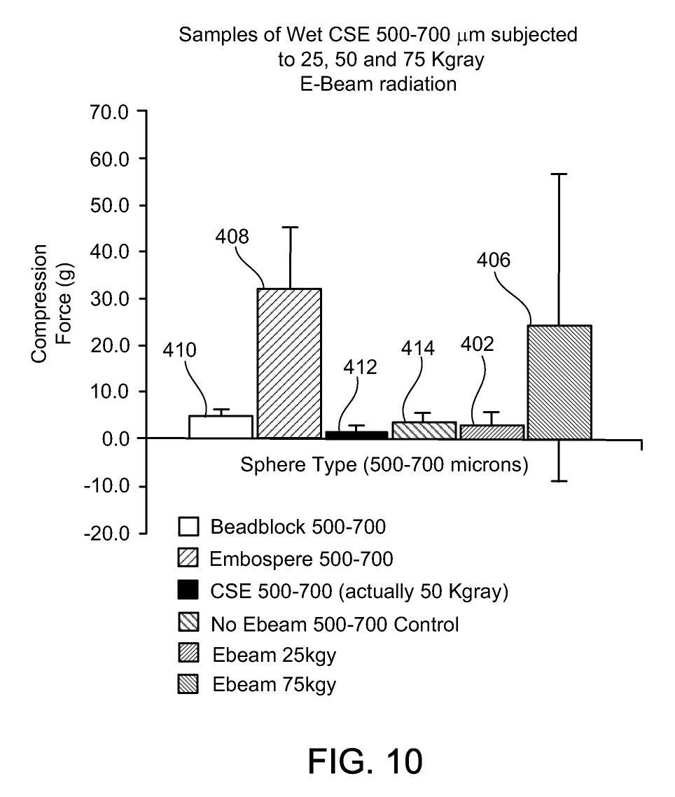

The presence and possible effects of nitrogen gas in wet Contour SE particles were also investigated. FIGS. 9 and 10 show compression force measurement results at 80% strain for Contour SE particles exposed to varying doses of electron beam radiation in a nitrogen-free environment and in an air-filled environment, respectively. In FIG. 9, blocks 302-306 represent the measured compression force values for Contour SE particles exposed to 25 kGy, 50 kGy, and 75 kGy electron beam radiation doses, respectively. Blocks 308 and 310 correspond, respectively, to compression force measurements for Embosphere and Beadblock particles, and are provided for comparative purposes. Block 312 indicates the measured compression force for Contour SE finished particles, which are exposed to a total electron beam radiation dose of 50 kGy (two separate 25 kGy doses during manufacturing to sterilize the particles). Block 314 indicates the measured compression force for Contour SE particles that received no dose of electron beam radiation.

The various blocks in FIG. 10—which indicate compression force measurements of Contour SE particles in a nitrogen-containing environment—are labeled to match corresponding blocks in FIG. 9 with the reference numbers increased by 100. Comparing the results of FIGS. 9 and 10, nitrogen did not appear to play a discernable role in affecting the cross-linking process of the Contour SE particles. However, it is anticipated that the presence of other gases may influence the effectiveness of cross-linking. For example, it is expected that oxygen gas may function as a radical initiator for the cross-linking process.

It has been generally observed from the results shown in FIGS. 5-9 that for particles having a relatively higher porosity (e.g., Type 1 and Contour SE) prior to cross-linking, the effects of cross-linking are not as pronounced. That is, the increase in measured compression force following cross-linking of such particles can be relatively small. In contrast, for particles having a relatively lower porosity (e.g., Type 2) prior to cross-linking, the effects of cross-linking can be more significant, and the increase in measured compression force following cross-linking can be relatively large.

Without wishing to be bound by theory, one possible explanation for this observation is that particle formulations with higher porosity include a larger number of pores filled with saline solution compared to lower porosity formulations. The saline within the pores functions as a sol within the gelled polymer regions, and reduces the efficiency of cross-linking by absorbing a significant portion of the cross-linking radiation. In addition, particles with higher porosity tend to have a “peach pit” structure that is more compressible than the structures of lower porosity particles. Cross-linking does not significantly reduce the compressibility of such particles because bonds formed during cross-linking cannot bridge the peach pit structures to impart rigidity to the particle.

Again without wishing to be bound by theory, another possible explanation for the observation that the effects of cross-linking are less significant for formulations with higher porosity is that the particles may undergo polymer leaching when sodium alginate and/or other sacrificial materials are removed. For example, a series of particles can be prepared from formulations that include poly(vinyl alcohol) (PVA) and sodium alginate in the following weight percentages: 10.5/0.65, 10.5/0.75, and 10.5/1.25. Although each type of particle is nominally formed so that it contains 10.5 weight percent PVA, it is known that some amount of PVA leaches out during the particle formation process. It is speculated that increasing the concentration of sodium alginate in the particles may assist in preventing PVA leaching. As a result, exemplary weight percentages for PVA and sodium alginate in the nominal particle formulations given above could be 9.5/0.65, 9.75/0.75, and 10.0/1.25 after formation of the particles.

In general, cross-linking can be considered complete when a particular number of bonds have been formed between a polymer chain and its neighboring chains. For example, a polymer structure can be considered cross-linked when one bond is formed between each polymer chain in the structure and at least one of its neighbors. It follows that the higher the concentration of polymer chains in a particle, the larger the dose of radiation required to cross-link the particle. As a result, given the effective compositions of the particles discussed above after PVA leaching, it is expected that a particular radiation dose is more effective at cross-linking the lower porosity particles—which also have lower PVA concentrations—than the higher porosity particles, so that the lower porosity particles have higher compression force values following cross-linking. It is also possible, for example, that to observe mechanical property changes in particles following cross-linking, a minimum concentration of PVA (and/or other cross-linkable polymer constituents) is required. As a result, when the alginate content in particle formulations is reduced for example, there may be a tradeoff between lower porosity which suggests improved cross-linking, and a polymer concentration in the particle formulation that is too small to yield significantly higher compression force values following cross-linking.

Other embodiments are in the claims.

1. A method, comprising:
providing a first particle having pores, the first particle having a first compression test value; and
partially cross-linking the first particle to form a second particle having pores, the second particle having a second compression test value that is larger than the first compression test value by 25% or more,
wherein a maximum dimension of the first particle is 5,000 microns or less, and a maximum dimension of the second particle is 5,000 microns or less.

2. The method of claim 1, wherein the second compression test value is larger than the first compression test value by 50% or more.

3. The method of claim 2, wherein the second compression test value is larger than the first compression test value by 100% or more.

4. The method of claim 3, wherein the second compression test value is larger than the first compression test value by 200% or more.

5. The method of claim 1, wherein partially cross-linking the first particle includes irradiating the first particle.

6. The method of claim 5, wherein irradiating the first particle comprises irradiating the first particle with electrons.

7. The method of claim 5, wherein irradiating the first particle comprises irradiating the first particle with gamma radiation.

8. The method of claim 1, wherein the first particle comprises vinyl alcohol monomer units, and the method comprises cross-linking at least some of the vinyl alcohol monomer units.

9. The method of claim 1, wherein the first particle comprises vinyl formal monomer units, and the method comprises cross-linking at least some of the vinyl formal monomer units.

10. The method of claim 6, wherein a dosage of the electron radiation is 25 kiloGray or more.

11. The method of claim 10, wherein the dosage of the electron radiation is 50 kiloGray or more.

12. The method of claim 11, wherein the dosage of the electron radiation is 75 kiloGray or more.

13. The method of claim 1, further comprising, before partially cross-linking the first particle, disposing a therapeutic agent in at least some of the pores of the first particle.

14. The method of claim 1, further comprising forming a plurality of the second particles, and forming a composition comprising a carrier solution and the plurality of the second particles.

15. The method of claim 14, further comprising treating a subject with the composition.

16. A method, comprising:
providing a particle having pores and a maximum dimension of 5,000 microns or less, the particle having a first compression test value;
selecting a desired compression test value for the particle; and
changing the compression test value of the particle from the first compression test value to the second compression test value,
wherein the second compression test value is at least 25% larger than the first compression test value.

17.-29. (canceled)

30. A method, comprising:
providing a first plurality of particles having pores, and disposing a therapeutic agent in at least some of the pores of the first plurality of particles; and
partially cross-linking the first plurality of particles to form a second plurality of particles having pores,
wherein the first plurality of particles has a first average compression test value, and the second plurality of particles has a second average compression test value that is larger than the first average compression test value by 25% or more.

31.-36. (canceled)

37. A method, comprising:
irradiating a first porous particle having a first compression test value to form a second porous particle having a second compression test value, the second compression test value being larger than the first compression test value.

38.-41. (canceled)
